(19) United States
(12) Patent Application Publication
(10) Pub. No.: US 2007/0029221 A1
(43) Pub. Date: Feb. 8, 2007

(54) Cervical collar bag

(76) Inventors: Nancy Gold, Niskayuna, NY (US); Inna Spektor, Niskayuna, NY (US); Ronald Dingman, Rotterdam, NY (US)

Correspondence Address: Nancy Gold;Tough Traveler LTD., 1012 State St., Schenectady, NY, 12307, US

(21) Appl. No.: 11/199,947

(22) Filed: Aug. 4, 2005

(51) Int. Cl. A61B 19/02; B65D 83/10

(52) U.S. Cl. 206/438; 206/363

(57) ABSTRACT

The present cervical collar bag invention is a bag that will allow emergency personnel to have the required cervical collars at hand and easily and quickly accessible and protected. Sized to fit the currently-available disposable adjustable cervical collars up to the quantity of eight, the present invention will also accommodate the older style cervical collars. One or more partitions will allow the means of separation of adult and pediatric collars or separation by other category or designation. The web grab handle, the ribbon handle at the zipper pulls, the two handles, the removable shoulder strap, and the extension of the tongue add to the convenience and importance of the invention, which may even be useful to non-emergency personnel for cervical collars or for other gear or usage.

This invention is a bag or a pouch to hold cervical collars, with the ability to protect them, to keep them easily accessible, and to separate particular sizes or types of cervical collars if desired.

### CROSS REFERENCE TO RELATED APPLICATIONS

There are no cross related applications.

### STATEMENT REGARDING FEDERALLY SPONSORED RESEARCH AND DEVELOPMENT

This invention did not have federally sponsored research and design.

### REFERENCE TO SEQUENCE LISTING, A TABLE, OR A COMPUTER PROGRAM LISTING APPENDIX

There is no appendix.

### BACKGROUND OF THE INVENTION

This cervical collar bag invention was conceived and developed to assist emergency and other personnel and persons when they are in situations requiring quick use of cervical collars. Cervical collars are important to emergency personnel in their assistance of both mildly injured people and people who have suffered extreme trauma.

Various states of the United States legally require ambulances and other emergency vehicles to have cervical collars on hand as cervical collars are often used to protect individuals who either have neck injuries or individuals who may have neck or related injuries. Currently cervical collars are either carried in a pocket of another emergency response bag such as a duffel or an oxygen bag, or they are carried separately in a duffel or other such independent bag or backpack. When they are carried in a pocket of another bag the size of the pocket may not be correct or the other items in the main portion of the bag may be overloaded, making the pocket smaller than intended. Also, the cervical collars may be carried separately in a bag that has no protection and may not have easy quick access to the collars. When carried in a bag without protection or with a too-small pocket—both of which might cause damaged cervical collars—and without easy, quick access to the collars, vital time may be lost in securing a non-damaged collar, in retrieving the collar from a cramped space, or in opening the bag. Also, carrying the bag may be cumbersome and therefore discouraging to the user who might ‘forget’ to bring it, and a non-specific bag might be confused with like bags or packs.

Currently there are no known specific cervical collar bags, although there are protective bags with unpadded side pockets for cervical collars [Tough Traveler® Re-Med® catalogue Mega-Duffel™]. There are many non-specific duffels, bags, or backpacks. Generally, bags or packs containing cervical collars are kept ready in buildings or temporary locations that house emergency resources, then they are carried by emergency personnel to the accident or other emergency. Sometimes these bags or packs are left in ambulances, fire engines, emergency helicopters, or other vehicles until needed at the trauma scene. With the many items that emergency personnel are responsible for, it is imperative that cervical collars not be crushed, left by the wayside, confused in bags with other items, or even lost in the melee, all of which are more likely with non-specific, non-protective, cumbersome bags or other containers of holders.

### BRIEF SUMMARY OF THE INVENTION

The current cervical collar bag invention provides emergency and other personnel a correctly-sized foam-padded, cloth material, bag for cervical collars, with easy quick access to those collars. The invention anticipates as few as one or two cervical collars (as it is believed required outfitting for emergency helicopters in New York State) and as many as eight cervical collars (generally ambulances are outfitted with six cervical collars). Currently there is the trend to use disposable adjustable collars, which the present invention accommodates, and it also accommodates the ‘older-fashioned’ non-adjustable, non-disposable collars as well as anticipates adjustments for any size changes that may occur in the future.

Other features of the present cervical collar bag invention include removable padded divider (to keep the adult-sized cervical collars separate from the child-sized or pediatric cervical collars), adjustable removable shoulder strap, top handles, end handle, double-zippered tongue opening, tongue handle, grab extension of the tongue, hook and loop sections, and reflective tape strips on all sides.

### BRIEF DESCRIPTION OF THE DRAWINGS

FIG. 1 Shows the closed top and one side of the cervical collar bag invention,

FIG. 2 shows the closed top and other side thereof,

FIG. 3 shows the bottom,

FIG. 4 shows one end,

FIG. 5 shows the other end,

FIG. 6 shows the closed top,

FIG. 7 shows the opened top,

FIG. 8a shows the separate partition,

FIG. 8b shows the un-partitioned inside,

FIG. 8c shows the partitioned inside.

FIG. 9 Shows a person using one hand to grip the ribbon handle of the cervical collar bag invention to open the bag, while using the other hand grip the web end handle thereof,

FIG. 10 shows a person using one hand to grip the grab-extension of the tongue to open the bag, while using the other hand to grip the web end handle.

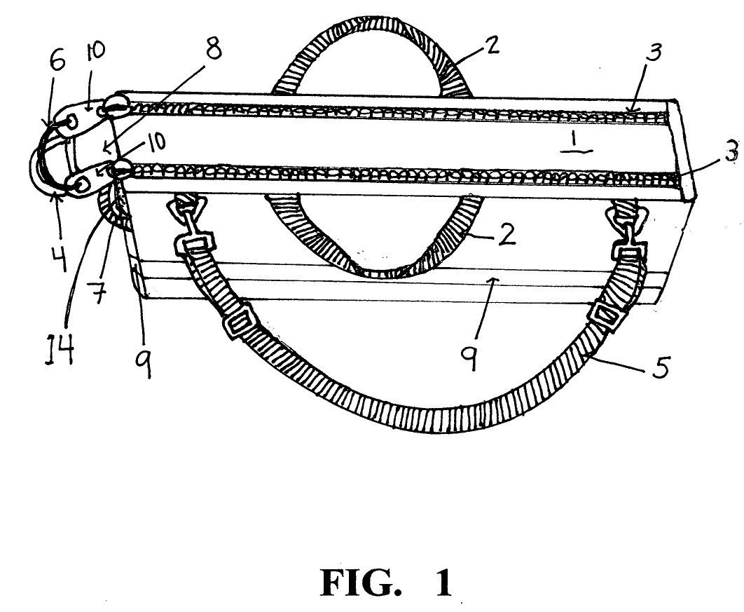

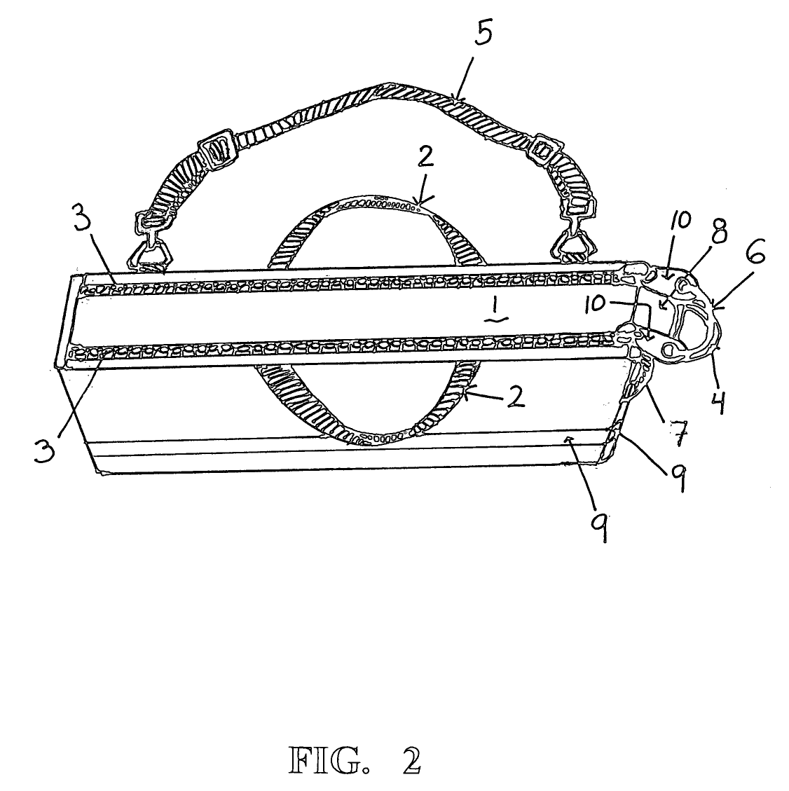

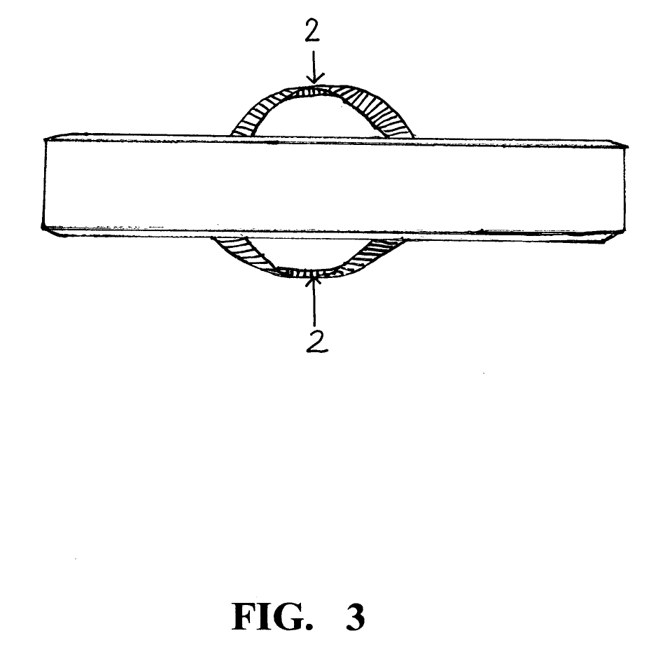

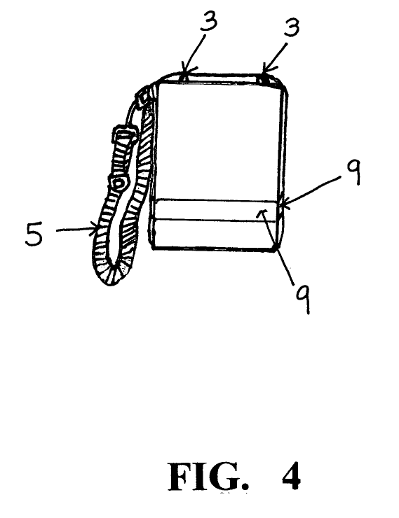

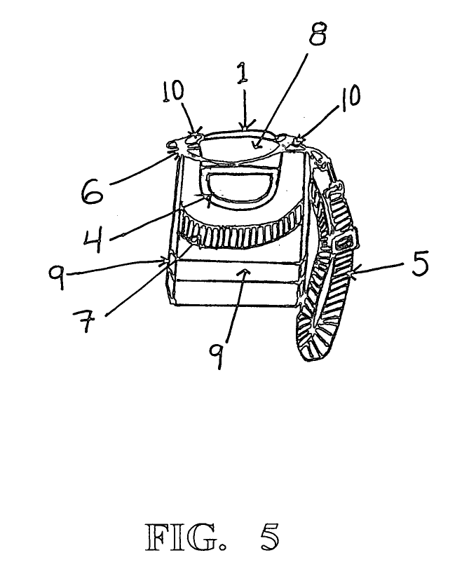

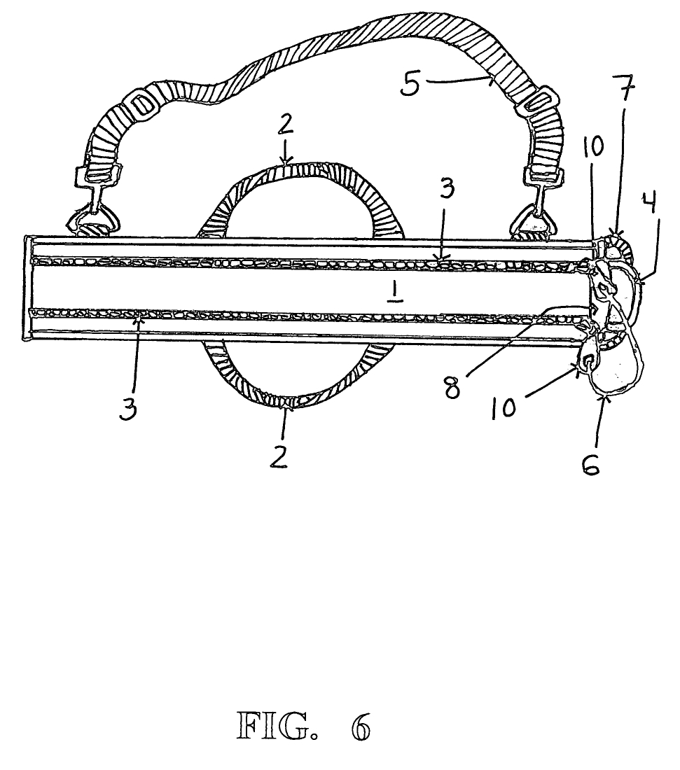

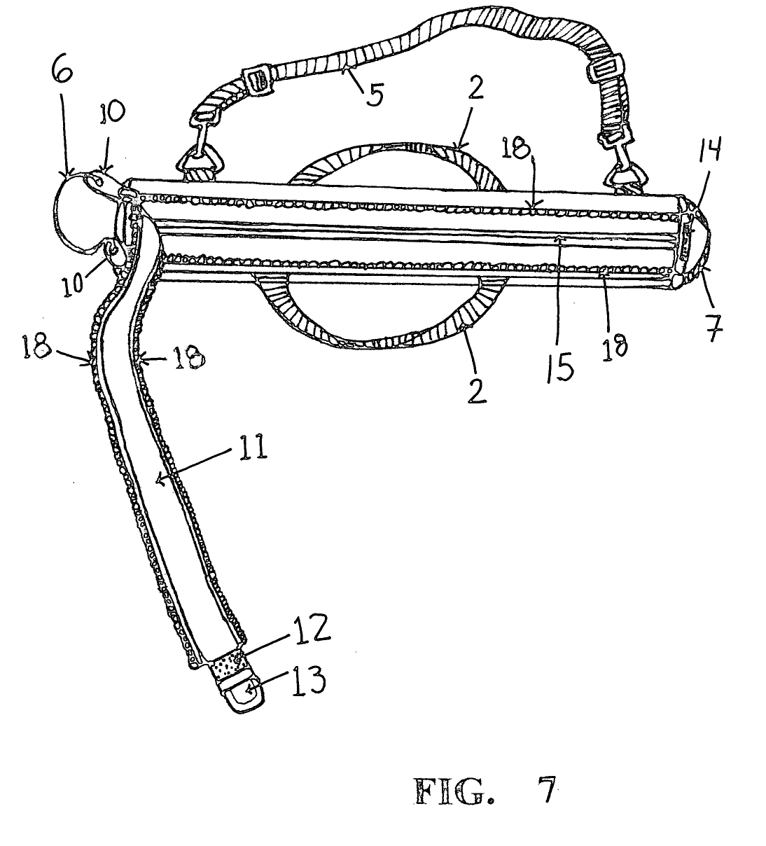

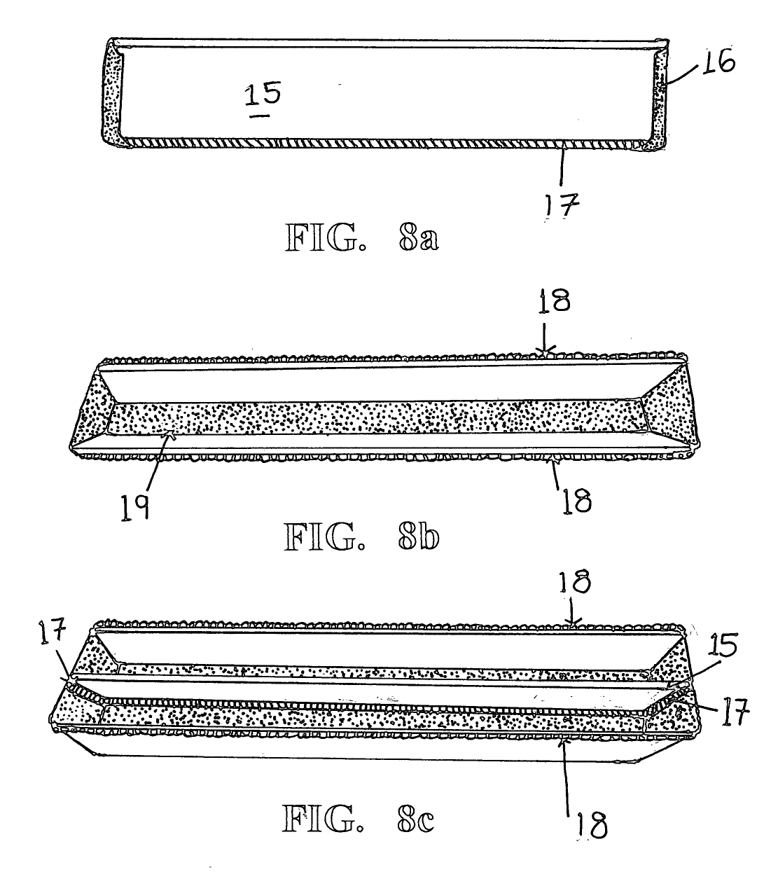

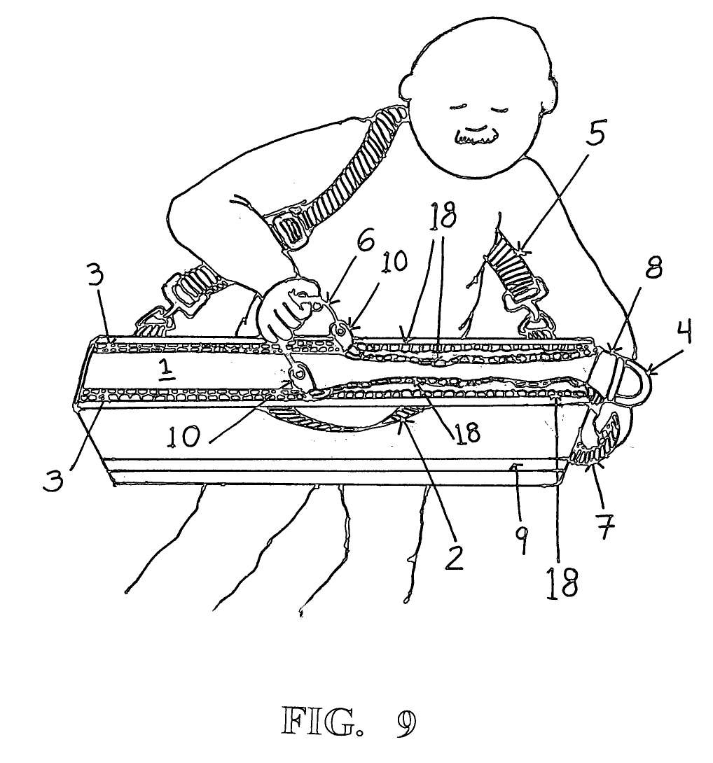

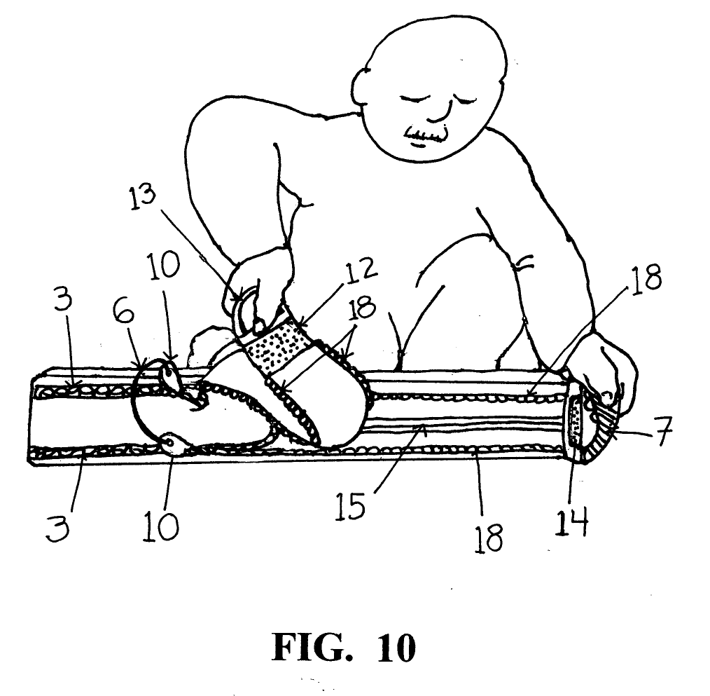

### DETAILED DESCRIPTION

The cervical collar bag invention as shown has a tongue 1 (topside), 11 (underside) cover at the top; web handles 2 at the top; removable shoulder strap 5; zippers 3 (closed), 18 (open) on two sides at the top; a zipper pull 10 at each zipper 3; a ribbon 6 connecting the zipper pulls 10; a web grab handle 7 on one end; a grab extension 4 (topside), 13 (underside) of the tongue 1 (topside), 11 (underside); reflective tape strips 9 on the two sides and the two ends; hook and loop 14 at one end which corresponds with hook and loop 12 which is sewn to the underside of the web 8 at end of the tongue 1 (topside), 11 (underside).

The cervical collar bag invention as shown has a removable foam-padded partition 15 which has a web 17 border on three sides, with hook and loop 16 sewn to the underside of that web border 17, and hook and loop 19 sewn to the invention inside at three places (the two ends and the bottom) so that the removable partition 15 can be attached or detached. The removable partition 15 has each side a different color to allow one type or size of the cervical collars, such as adult, to be separated from another type or size of cervical collars, such as pediatric.

Although it is thought that the present embodiment as described is the optimum embodiment for emergency personnel needs, alternative embodiments of this cervical collar bag invention can be imagined by one practiced in the art. An example such as a backpack using detachable, hide-away, or integral shoulder straps may be imagined. Also, modifications such as backpack straps as described may be on the present invention. Modifications in the size of the invention and/or in the number of partitions may be made to accommodate different sizes or different quantities of cervical collars should the need arise. Different materials may be used, such as one might imagine even aluminum as casing, with partitions conceivably of mesh netting or even an interior mesh pocket open at the top; cord may be used instead of ribbon, and other material changes might be imagined. To simplify, there might be no handles, and to aggrandize there might be outer pockets for spare disposable gloves, tracheal equipment, and/or other emergency items. Reflective tape strips might be omitted or might take alternative forms, for example reflective threads or a hide-away reflective tape configuration. Alternatives to the tongue cover as used in the current embodiment might be either a slit opening, a hook and loop closure, a one-zipper opening, a quick-release buckle opening, or additional configurations as imaginable by one practiced in the art. An additional embodiment might be a two-zipper opening with one compartment below each zipper. It is even possible that an embodiment with no top can be imagined. The present embodiment of the cervical collar bag invention could be sewn-on to another emergency equipment bag or otherwise attached, perhaps with hook and loop, or perhaps with quick release buckles, to such a bag or backpack.

We claim:

1. A container comprising at least one compartment adapted to hold at least one cervical collar.

2. The container of claim 1, further comprising a device for partially closing the at least one compartment.

3. The container of claim 1, including a shoulder strap.

4. The container of claim 1, including a handle.

5. The container of claim 1, further comprising at least one partition, whereby the at least one compartment is adapted to hold a plurality of cervical collars.

6. The container of claim 1, further comprising a device for closing the at least one compartment.

7. The container of claim 5, wherein the plurality of cervical collars includes a pediatric cervical collar.

8. The container of claim 5, wherein the plurality of cervical collars includes an adult cervical collar.

9. The container of claim 5, wherein the at least one partition is removable.

10. The container of claim 5, wherein the at least one partition includes at least two colors of categorizing each of the plurality of cervical collars.

11. The container of claim 5, wherein the at least one partition includes at least two material pieces of categorizing each of the plurality of cervical collars.

12. The container of claim 6, wherein the device includes at least one of the following: a zipper, a tongue cover, and hook and loop.

13. The container of claim 6, wherein the device includes at least one of the following: a cover and hook and loop.

14. The container of claim 6, wherein the device includes at least one of the following: a zipper, a tongue cover, hook and loop, and an extension piece on the tongue cover.

15. The container of claim 6, wherein the device includes at least one of the following: a zipper, a tongue cover, hook and loop, an extension piece on the tongue cover, and a grab handle on the opposing end of the container.

16. The container of claim 6, wherein the device includes at least one of the following: a zipper, a tongue cover, hook and loop, and a grab handle connected to a zipper pull.

17. The container of claim 6, wherein the device includes at least one of the following: a tongue cover, hook and loop, and an extension piece on the tongue cover.

18. The container of claim 6, wherein the device includes at least one of the following: a cover and a quick-release buckle.

19. The container of claim 6, wherein the device consists of a cover and elastic.
